(19) United States
(12) Patent Application Publication
(10) Pub. No.: US 2014/0370559 A1
(43) Pub. Date: Dec. 18, 2014

(54) FERMENTATION OF WASTE GASES

(71) Applicant: LanzaTech New Zealand Limited, Auckland (NZ)

(72) Inventors: Simon David Oakley, Auckland (NZ); Joss Anton Coombes, Roselle, IL (US); Sean Dennis Simpson, Auckland (NZ); Bjorn Daniel Heijstra, Auckland (NZ); Michael Anthony Schultz, Roselle, IL (US); Sean Molloy, Auckland (NZ)

(73) Assignee: LanzaTech New Zealand Limited, Auckland (NZ)

(21) Appl. No.: 14/214,187

(22) Filed: Mar. 14, 2014

Related U.S. Application Data
(63) Continuation of application No. 13/640,292, filed on Oct. 9, 2012, now abandoned.
(60) Provisional application No. 61/331,237, filed on May 4, 2010.

(51) Int. Cl. C12P 7/56; C12P 7/18; C12P 7/54; C12P 7/06

(52) U.S. Cl. CPC C12P 7/56; C12P 7/065; C12P 7/18; C12P 7/54
USPC 435/139; 435/157; 435/136; 435/161; 435/158; 435/140

(57) ABSTRACT

The invention relates to the microbial fermentation of gaseous substrates to produce one or more products. The invention relates to the microbial fermentation of a gaseous substrate derived from the conversion of a biogas stream. The invention relates to the conversion of a biogas stream comprising methane to a gaseous substrate comprising CO or CO plus H2, and the production of one or more products from the microbial fermentation of said gaseous substrate.

### CROSS REFERENCE TO RELATED APPLICATIONS

This application is a continuation of pending U.S. application Ser. No. 13/640,292 filed on 9 Oct. 2012 which in turn is a National Stage of International Application No. PCT/NZ2011/000064, filed on 4 May 2011, which claims the benefit of the priority date of U.S. Provisional Application No. 61/331,237 filed 4 May 2010. The content of all of which applications mentioned above are incorporated herein by reference in their entirety.

### FIELD OF THE INVENTION

This invention relates to systems and methods for improving overall carbon capture and/or improving overall efficiency in processes including microbial fermentation. In particular, the invention relates to improving carbon capture and/or improving efficiency in processes including microbial fermentation of a reformed substrate stream comprising CO and H2.

### BACKGROUND OF THE INVENTION

Ethanol is rapidly becoming a major hydrogen-rich liquid transport fuel around the world. Worldwide consumption of ethanol in 2005 was an estimated 12.2 billion gallons. The global market for the fuel ethanol industry has also been predicted to grow sharply in future, due to an increased interest in ethanol in Europe, Japan, the USA, and several developing nations.

For example, in the USA, ethanol is used to produce E10, a 10% mixture of ethanol in gasoline. In E10 blends the ethanol component acts as an oxygenating agent, improving the efficiency of combustion and reducing the production of air pollutants. In Brazil, ethanol satisfies approximately 30% of the transport fuel demand, as both an oxygenating agent blended in gasoline, and as a pure fuel in its own right. Also, in Europe, environmental concerns surrounding the consequences of Green House Gas (GHG) emissions have been the stimulus for the European Union (EU) to set member nations a mandated target for the consumption of sustainable transport fuels such as biomass derived ethanol.

The vast majority of fuel ethanol is produced via traditional yeast-based fermentation processes that use crop derived carbohydrates, such as sucrose extracted from sugarcane or starch extracted from grain crops, as the main carbon source. However, the cost of these carbohydrate feed stocks is influenced by their value as human food or animal feed, while the cultivation of starch or sucrose-producing crops for ethanol production is not economically sustainable in all geographies. Therefore, it is of interest to develop technologies to convert lower cost and/or more abundant carbon resources into fuel ethanol.

CO is a major, low cost, energy-rich by-product of the incomplete combustion of organic materials such as coal or oil and oil derived products. For example, the steel industry in Australia is reported to produce and release into the atmosphere over 500,000 tonnes of CO annually. Additionally or alternatively, CO rich gas streams (syngas) can be produced by gasification of carbonaceous materials, such as coal, petroleum and biomass. Carbonaceous materials can be converted into gas products including CO, CO2, H2 and lesser amounts of CH4 by gasification using a variety of methods, including pyrolysis, tar cracking and char gasification. Syngas can also be produced in a steam reformation process, such as the steam reformation of methane or natural gas. Methane can be converted to hydrogen and carbon monoxide and/or carbon dioxide by methane reformation in the presence of a metal catalyst. For example, steam reformation of methane occurs as follows:

CH₄+H₂O→CO+3H₂ (1)

CO+H₂O→CO₂+H₂ (2)

This process accounts for a substantial portion of the hydrogen produced in the world today. Attempts to use the hydrogen produced in the above reactions in fuel cell technology have been largely unsuccessful, due to the presence of carbon monoxide, which typically poisons fuel cell catalysts. Other catalytic processes may be used to convert gases consisting primarily of CO and/or CO and hydrogen (H₂) into a variety of fuels and chemicals. Micro-organisms may also be used to convert these gases into fuels and chemicals. These biological processes, although generally slower than chemical reactions, have several advantages over catalytic processes, including higher specificity, higher yields, lower energy costs and greater resistance to poisoning.

The ability of micro-organisms to grow on CO as a sole carbon source was first discovered in 1903. This was later determined to be a property of organisms that use the acetyl coenzyme A (acetyl CoA) biochemical pathway of autotrophic growth (also known as the Woods-Ljungdahl pathway and the carbon monoxide dehydrogenase/acetyl CoA synthase (CODH/ACS) pathway). A large number of anaerobic organisms including carboxydotrophic, photosynthetic, methanogenic and acetogenic organisms have been shown to metabolize CO to various end products, namely CO₂, H₂, methane, n-butanol, acetate and ethanol. While using CO as the sole carbon source, all such organisms produce at least two of these end products.

Anaerobic bacteria, such as those from the genus Clostridium, have been demonstrated to produce ethanol from CO, CO₂ and H₂ via the acetyl CoA biochemical pathway. For example, various strains of Clostridium ljungdahlii that produce ethanol from gases are described in WO 00/68407, EP 117309, U.S. Pat. Nos. 5,173,429, 5,593,886, and 6,368,819, WO 98/00558 and WO 02/08438. The bacterium Clostridium autoethanogenum sp is also known to produce ethanol from gases (Abrini et al., Archives of Microbiology 161, pp 345-351 (1994)).

However, ethanol production by micro-organisms by fermentation of gases is typically associated with co-production of acetate and/or acetic acid. As some of the available carbon is typically converted into acetate/acetic acid rather than ethanol, the efficiency of production of ethanol using such fermentation processes may be less than desirable. Also, unless the acetate/acetic acid by-product can be used for some other purpose, it may pose a waste disposal problem. Acetate/acetic acid is converted to methane by micro-organisms and therefore has the potential to contribute to GHG emissions.

WO2007/117157 and WO2008/115080, the disclosure of which are incorporated herein by reference, describe processes that produce alcohols, particularly ethanol, by anaerobic fermentation of gases containing carbon monoxide. Acetate produced as a by-product of the fermentation process described in WO2007/117157 is converted into hydrogen gas and carbon dioxide gas, either or both of which may be used in the anaerobic fermentation process.

The fermentation of gaseous substrates comprising CO, to produce products such as acids and alcohols, typically favours acid production. Alcohol productivity can be enhanced by methods known in the art, such as methods described in WO2007/117157, WO2008/115080, WO2009/022925 and WO2009/064200, which are fully incorporated herein by reference.

U.S. Pat. No. 7,078,201 and WO 02/08438 also describe fermentation processes for producing ethanol by varying conditions (e.g. pH and redox potential) of the liquid nutrient medium in which the fermentation is performed. As disclosed in those publications, similar processes may be used to produce other alcohols, such as butanol.

Microbial fermentation of CO in the presence of H₂ can lead to substantially complete carbon transfer into an alcohol. However, in the absence of sufficient H₂, some of the CO is converted into alcohol, while a significant portion is converted to CO₂ as shown in the following equations:

6CO+3H₂O→C₂H₅OH+4CO₂

12H₂+4CO₂→2C₂H₅OH+6H₂O

The production of CO₂ represents inefficiency in overall carbon capture and if released, also has the potential to contribute to Green House Gas emissions. Furthermore, carbon dioxide and other carbon containing compounds, such as methane, produced during a gasification process may also be released into the atmosphere if they are not consumed in an integrated fermentation reaction.

It is an object of the present invention to provide system(s) and/or method(s) that overcomes disadvantages known in the art and provides the public with new methods for the optimal production of a variety of useful products.

### SUMMARY OF THE INVENTION

In accordance with a first aspect, the invention provides a method for producing products from a biogas stream, the method comprising:

1) conversion of at least a portion of the biogas stream comprising methane to a substrate stream comprising CO and H2;
2) anaerobic fermentation of at least a portion of the CO and optionally H2 from step (1) to produce products.

In particular embodiments of the invention, biogas is converted to a substrate stream comprising CO and H2 by catalytic oxidation. In particular embodiments, at least portions of components such as H2S, CO2, O2 and/or N2 are removed from the biogas prior to catalytic oxidation. Those skilled in the art will appreciate methods for removal of one or more components from a biogas stream. Additionally or alternatively, a methane component of the biogas stream is enriched prior to catalytic oxidation.

In particular embodiments, at least a portion of a methane component of a biogas stream is converted to a substrate stream comprising CO and H2 by catalytic oxidation. In certain embodiments, catalytic oxidation is conducted at 700-1100° C. in the presence of a Ni catalyst.

In one embodiment a methane component of a biogas stream is converted to a substrate stream comprising CO and H2 by a steam reforming reaction having the following stoichiometry;

CH₄+H₂O->3H₂+CO

The steam reforming process is conducted at 700-1100° C. in the presence of a nickel-alumina catalyst.

In one embodiment of the invention the biogas stream is blended with CO₂ to obtain a CH₄:CO₂ ratio of around 1:1 or around 2:1 or around 3:1.

In a second aspect, the invention provides a method for producing products including acids and/or alcohols from a methane stream, the method comprising:

1) conversion of a least a portion of the methane stream to a substrate stream comprising CO and H2;
2) anaerobic fermentation of at least a portion of the CO and optionally H2 from step (1) to produce products.

According to a third aspect, the invention provides a method of improving overall efficiency of a fermentation, the method including:

1) converting methane to a substrate stream comprising CO and H2;
2) blending CO and/or H2 to the substrate stream to optimise the CO:H2 ratio;
3) anaerobic fermentation of at least a portion of CO and optionally H2 from step (2) to produce products.

In particular embodiments the blended stream may substantially comprise CO and H2 in the following molar ratios: at least 20:1, at least 10:1, at least 5:1, at least 3:1, at least 2:1, at least 1:1 or at least 1:2 (CO:H2).

In particular embodiments of the second and third aspects, the methane is derived from biogas comprising methane.

In particular embodiments, CO blended to the substrate stream comprising CO and H2 is a waste stream derived from an industrial process. In particular embodiments, the industrial waste stream is steel mill off gas comprising CO.

In particular embodiments of the various preceding aspects, the anaerobic fermentation produces products including acid(s) and alcohol(s) from CO and optionally H2. In particular embodiments, the anaerobic fermentation is conducted in a bioreactor, wherein one or more microbial cultures convert CO and optionally H2 to products including acid(s) and/or alcohol(s). In certain embodiments, the product is ethanol.

In particular embodiments, the microbial culture is a culture of carboxydotrophic bacteria. In certain embodiments, the bacteria is selected from Clostridium, Moorella and Carboxydothermus. In particular embodiments, the bacterium is Clostridium autoethanogenum.

According to various embodiments of the invention, the substrate stream and/or the blended stream provided to the fermentation will typically contain a major proportion of CO, such as at least about 20% to about 95% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when significant amounts of H₂ and optionally CO₂ are present.

According to another aspect, the invention provides a system for producing products by microbial fermentation, the system including:

1) a catalytic oxidation stage, wherein methane and/or biogas is converted to a substrate stream comprising CO and H2;
2) means to pass the substrate stream comprising CO and H2 to a bioreactor;
3) a bioreactor configured to convert at least a portion of the substrate stream to products by microbial fermentation.

A gas separation stage may optionally remove at least portions of one or more components from a gas stream prior to catalytic oxidation.

In particular embodiments, the system comprises means for determining whether the substrate stream comprising CO and H2 has a desired composition. Any known means may be used for this purpose.

In particular embodiments, the system further includes blending means configured to blend CO and/or H2 to the substrate stream prior to passing to the bioreactor. In particular embodiments, the system comprises means for diverting gas away from the bioreactor if the means for determining determines that the gas does not have the desired composition.

In particular embodiments of the invention, the system includes means for heating and/or cooling the various streams passed between various stages of the system. Additionally or alternatively, the system includes means for compressing at least portions of the various streams passed between various stages of the system.

In particular embodiments of the invention, the biogas comprising methane is produced in one or more digester and the system includes means of passing the biogas to the catalytic oxidation stage. In particular embodiments, the biogas is passed via a gas separation and/or methane enrichment stage. In particular embodiments, the biogas is produced in a single digester configured to digest biodegradable material transported to the digester. In another embodiment, the biogas is produced in multiple remote digesters, and the biogas passed to the catalytic oxidation stage. Those skilled in the art will appreciate means for transporting biodegradable material to the digester. Those skilled in the art will also appreciate means for passing biogas from multiple remote digesters to a catalytic oxidation stage.

Although the invention is broadly as defined above, it is not limited thereto and also includes embodiments of which the following description provides examples.

### BRIEF DESCRIPTION OF THE DRAWINGS

The invention will now be described in detail with reference to the accompanying Figures in which:

FIG. 1: is a schematic representation of a system according to one embodiment of the invention, including a methane reformer.

FIG. 2: is a schematic representation of a system according to one embodiment of the invention, including blending means.

FIG. 3: is a graphical representation of CO₂ concentration (%) in accordance with Example 2.

FIG. 4: is a graphical representation of CO concentration (%) in accordance with Example 2

FIG. 5: is a graphical representation of CO₂ concentration (%) in accordance with Example 2.

FIG. 6: is a graphical representation of CO concentration (%) in accordance with Example 2.

### DETAILED DESCRIPTION OF THE INVENTION

Biogas comprising methane is produced in large quantities by anaerobic digestion of biodegradable carbonaceous materials. Biogas typically comprises 50-75% methane, which is commonly burned to utilise the energy. It has been recognised that the hydrogen and CO produced from reformation of methane derived from biogas can be converted to products, such as acids and alcohols by anaerobic fermentation. In accordance with particular methods of the invention, at least a portion of the methane component of biogas is converted to carbon monoxide and hydrogen by methane reformation. The resulting stream comprising CO and H2 is in turn converted to products such as acids and alcohols by microbial fermentation in a bioreactor. Thus, in accordance with particular embodiments, biogas is converted into transportable liquid products.

In another embodiment, there is provided a method for producing products, such as acid(s) and/or alcohol(s) from biogas, the method comprising:

1) conversion of at least a portion of the biogas to a stream comprising CO and H2;
2) anaerobic fermentation of at least a portion of the CO and optionally H2 from step (1) to produce products.

It is further recognised that the efficiency of the fermentation step can be improved by optimising the CO:H2 ratio of the substrate stream. For example, in particular embodiments of the invention, the fermentation produces ethanol according to:

2CO+4H₂→CH₃CH₂OH+H₂O

The CO:H2 ratio of the reformed methane stream can be altered to increase the overall CO content (up to 1:1) by changing reformation parameters. For example, methane can be reformed in the presence of oxygen and CO2 in a process known as autothermal reforming:

2CH₄+O₂+CO₂→3H₂+3CO+H₂O

Thus, a stream comprising CO and H2 of a desired composition can be produced from biogas by selecting desired reformation parameters. In accordance with particular embodiments, a stream comprising CO and H2 of a desired composition is provided to a microbial culture in a bioreactor, where at least a portion of the stream is converted to products, such as ethanol, by microbial fermentation.

Additionally or alternatively, a stream with a desired CO and H2 composition may be produced by blending the reformed methane stream comprising CO and H2 with CO and/or H2 from an alternative source. For example, CO is produced as a waste product in a variety of industrial processes, such as steel production. In particular embodiments, the CO derived from such industrial processes can be blended with the reformed methane stream comprising CO and H2 to produce a stream with a desired CO and H2 composition and passed to the bioreactor for conversion into products.

### DEFINITIONS

Unless otherwise defined, the following terms as used throughout this specification are defined as follows:

The terms “carbon capture” and “overall carbon capture” refer to the efficiency of conversion of a carbon source, such as a feedstock, into products. For example, the amount of carbon in a woody biomass feedstock converted into useful products, such as alcohol.

The term “syngas” refers to a gas mixture that contains at least a portion of carbon monoxide and hydrogen produced by gasification and/or reformation of a carbonaceous feedstock.

The term “biogas” refers to a gas mixture that contains at least a portion of methane produced by anaerobic digestion of biodegradable material(s).

The term “substrate comprising carbon monoxide” and like terms should be understood to include any substrate in which carbon monoxide is available to one or more strains of bacteria for growth and/or fermentation, for example.

“Gaseous substrates comprising carbon monoxide” include any gas which contains carbon monoxide. The gaseous substrate will typically contain a significant proportion of CO, preferably at least about 5% to about 95% CO by volume.

The term “bioreactor” includes a fermentation device consisting of one or more vessels and/or towers or piping arrangements, which includes the continuous stirred tank reactor (CSTR), an immobilised cell reactor, a gas-lift reactor, a bubble column reactor (BCR), a membrane reactor, such as a Hollow Fibre Membrane Bioreactor (HFMBR) or a trickle bed reactor (TBR), or other vessel or other device suitable for gas-liquid contact.

The term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. In addition, the term “acetate” includes both acetate salt alone and a mixture of molecular or free acetic acid and acetate salt, such as the mixture of acetate salt and free acetic acid present in a fermentation broth as described herein.

The term “desired composition” is used to refer to the desired level and types of components in a substance, such as, for example, of a gas stream. More particularly, a gas is considered to have a “desired composition” if it contains a particular component (e.g. CO and/or H₂) and/or contains a particular component at a particular level and/or does not contain a particular component (e.g. a contaminant harmful to the micro-organisms) and/or does not contain a particular component at a particular level. More than one component may be considered when determining whether a gas stream has a desired composition.

The term “stream” is used to refer to a flow of material into, through and away from one or more stages of a process, for example, the material that is fed to a bioreactor and/or an optional CO₂ remover. The composition of the stream may vary as it passes through particular stages. For example, as a stream passes through the bioreactor, the CO content of the stream may decrease, while the CO₂ content may increase. Similarly, as the stream passes through the CO₂ remover stage, the CO₂ content will decrease.

Unless the context requires otherwise, the phrases “fermenting”, “fermentation process” or “fermentation reaction” and the like, as used herein, are intended to encompass both the growth phase and product biosynthesis phase of the process.

The terms “increasing the efficiency”, “increased efficiency” and the like, when used in relation to a fermentation process, include, but are not limited to, increasing one or more of: the rate of growth of micro-organisms in the fermentation, the volume or mass of desired product (such as alcohols) produced per volume or mass of substrate (such as carbon monoxide) consumed, the rate of production or level of production of the desired product, and the relative proportion of the desired product produced compared with other by-products of the fermentation, and further may reflect the value (which may be positive or negative) of any by-products generated during the process.

While certain embodiments of the invention, namely those that include the production of ethanol by anaerobic fermentation using CO and H2 as the primary substrate, are readily recognized as being valuable improvements to technology of great interest today, it should be appreciated that the invention is applicable to production of alternative products such as other alcohols and the use of alternative substrates, particularly gaseous substrates, as will be known by persons of ordinary skill in the art to which the invention relates upon consideration of the instant disclosure. For example, gaseous substrates containing carbon dioxide and hydrogen may be used in particular embodiments of the invention. Further, the invention may be applicable to fermentations to produce acetate, butyrate, propionate, caproate, ethanol, propanol, and butanol, and hydrogen. By way of example, these products may be produced by fermentation using microbes from the genus Moorella, Clostridia, Ruminococcus, Acetobacterium, Eubacterium, Butyribacterium, Oxobacter, Methanosarcina, Methanosarcina, and Desulfotomaculum.

### Biogas Production

Biogas is produced by anaerobic digestion of biodegradable feedstock such as biomass, manure, sewage, municipal waste, green waste and energy crops. In addition, biogas (or landfill gas) is produced by wet organic waste decomposing under anaerobic conditions in a land fill. The composition of biogas varies depending on the origin of the anaerobic digestion process. For example, land fill gas typically comprises methane concentrations of around 50%, whereas more advanced waste treatment technologies familiar to those skilled in the art produce biogas with 55-75% methane. Biogas also typically comprises additional components such as CO2 (20-45%), N2 (0-10%), H2 (0-1%), H2S (0-3%) and/or O2 (0-2%). Biogas can be burned to produce energy and/or electricity. Additionally or alternatively, the methane content of the biogas can be enriched using a biogas upgrader to produce biomethane. A biogas upgrader is a facility that can be used to concentrate the methane in biogas to natural gas standards. The methane is enriched in biomethane by removing components such as CO2, N2, H2, H2S and/or O2.

Biogas is typically produced in a sealed digester chamber under anaerobic conditions. For example, biomass can be added to a sealed chamber, wherein microbes digest the organic matter to produce biogas over time. Landfill biogas is produced in a similar manner. However, landfill waste is maintained under anaerobic conditions by piling further waste over existing waste, such that the existing waste is compressed resulting in an anaerobic environment for microbial digestion. Water and/or heat may be added or removed from the digestion in order to optimise digester conditions.

In accordance with the invention, biogas can be generated in a central location where a feedstock or a combination of feedstocks are available or are easily transportable to the central location. For example, biogas can be generated at a landfill site where municipal waste is dumped or a sewage treatment facility. Additionally or alternatively, lesser amounts of biogas can be produced in a plurality of remote locations, such as manure pits in farms, and piped to one or more locations for use in accordance with the methods of the invention.

### Biogas Conversion

In accordance with the methods of the invention, at least a portion of biogas is converted to a reformed substrate stream comprising CO and H2 by catalytic oxidation. In particular embodiments, methane derived from biogas is converted to CO and H2 in the presence of a metal catalyst at elevated temperature. The most common catalytic oxidation process is steam reforming, wherein methane and steam are reformed to CO and H2 at 700-1100° C. in the presence of a nickel catalyst. The stoichiometry of the conversion is as follows:

CH₄+H₂O→CO+3H₂

Additionally or alternatively, autothermal reforming can be used to partially oxidise methane in the presence of oxygen at elevated temperature and pressure as follows:

2CH₄+O₂+CO₂→3H₂+3CO+H₂O

2CH₄+O₂+H₂O→5H₂+2CO

Dry reforming takes advantage of the significant portion of CO2 present in biogas to produce carbon monoxide and hydrogen as follows:

CH₄+CO₂→2CO+2H₂

In accordance with the methods of the invention, the CO and H2 produced in the catalytic oxidation are used as a substrate stream, which is passed to a bioreactor to be converted to products by microbial fermentation.

In one embodiment of the invention, the biogas comprising methane is blended with CO₂ to obtain a CH₄:CO₂ ratio of around 1:1, or around 2:1 or around 3:1.

In particular embodiments of the invention, biogas can be converted to a reformed substrate stream comprising CO and H2 by catalytic oxidation without additional processing steps. However, as noted previously, biogas can contain components such as CO2, N2, H2S, and/or O2, any or all of which may adversely affect the catalytic oxidation process. For example, hydrogen sulfide may poison metal catalysts typically used in the catalytic oxidation process. For example, levels of H2S above 50 ppm are reported to poison a nickel catalyst at elevated temperature. As such, in accordance with particular methods of the invention, a biogas stream is treated such that the H2S content is less than 50 ppm prior to catalytic oxidation.

Furthermore, while CO2 and O2 may be used as reactants in the catalytic oxidation process, the presence of these components can affect the overall CO:H2 ratio of the substrate stream. Additionally, while N2 is unlikely to adversely affect the reformation of methane, the overall efficiency of the process will be reduced as additional gas must be heated and compressed.

As such, in particular embodiments of the invention, components such as CO2, N2, H2S and/or O2 are removed from biogas to produce an enriched biomethane stream suitable for catalytic oxidation. Such components can be removed using standard conditioning methodology in multiple unit operations. Those skilled in the art will be familiar with unit operations for removal of at least a portion of CO2, N2, H2S and/or O2. However, by way of example, H2S and/or CO2 (and other acidic gases) can be selectively removed from a gas stream using gas removal technologies known to those skilled in the art, such as Sulfurex™, Rectisol™, Genosorb™ or Selexol™.

Additionally or alternatively, technologies based on aqueous and/or water scrubbers can effectively remove CO2 and sulfides, thus increasing the CH₄ content of biogas. For example, biogas can be compressed to around 5-15 bar and passed into the bottom of a scrubbing column where it is contacted with a countercurrent of water. The columns are typically filled with packing to create a large wetted contact surface area. CO2 and H2S are well solubilised in water, so the resulting gas exiting the column is substantially enriched in methane. Typically, the exiting methane is dried to remove water vapour from the gas.

Pressure swing adsorption (PSA) is another method which can be used to enrich the methane component of the biogas stream. Biological desulphurization, using impregnated activated carbon, iron hydroxide or oxide and using sodium hydroxide for scrubbing are all effective methods of removing the H2S. Removal of other contaminants in the form of trace gases can be achieved with halogenated hydrocarbon removal, siloxane removal and removal of oxygen, nitrogen and water from the biogas. Other methods used in gas separation and enrichment such as membrane separation and cryogenic separation may also be used and are detailed in PCT/NZ2008/000275, which is fully incorporated herein by reference.

In particular embodiments of the invention, the composition of the biogas can be optimised by blending additional components from one or more alternative sources prior to catalytic oxidation. For example, it can be desirable to provide a substrate stream with a particular CO:H2 ratio to the bioreactor for microbial fermentation. In particular embodiments of the invention, autothermal reformation converts methane to CO and H2 in the presence of O2 and H2O or CO2. One or more of these additional components can be blended into the gas stream prior to reformation. Those skilled in the art will appreciate suitable component volumes to be blended into the biogas stream in order to optimise a desired reformed substrate stream comprising CO and H2.

In accordance with the methods of the invention, the resultant reformed substrate stream comprising CO and H2 can be passed directly to a bioreactor for conversion to products by microbial fermentation. However, in particular embodiments, one or more additional processing steps, such as gas cooling, particulate removal, gas storage, buffering, compression may be necessary to improve overall efficiency of the process. Examples of apparatus suitable for achieving one or more of the optional additional steps are detailed in PCT/NZ2008/000275, which is fully incorporated herein by reference.

### Blending of Streams

As noted previously, it may be desirable to blend a reformed substrate stream comprising CO and H2 with one or more further streams in order to improve efficiency, alcohol production and/or overall carbon capture of the fermentation reaction. Without wishing to be bound by theory, in some embodiments of the present invention, carboxydotrophic bacteria convert CO to ethanol according to the following:

6CO+3H₂O→C₂H₅OH+4CO₂

However, in the presence of H2, the overall conversion can be as follows:

6CO+12H₂→3C₂H₅OH+3H₂O

Accordingly, streams with high CO content can be blended with reformed substrate streams comprising CO and H2 to increase the CO:H2 ratio to optimise fermentation efficiency. By way of example, industrial waste streams, such as off-gas from a steel mill have a high CO content, but include minimal or no H2. As such, it can be desirable to blend one or more streams comprising CO and H2 with the waste stream comprising CO, prior to providing the blended substrate stream to the fermenter. The overall efficiency, alcohol productivity and/or overall carbon capture of the fermentation will be dependent on the stoichiometry of the CO and H2 in the blended stream. However, in particular embodiments the blended stream may substantially comprise CO and H2 in the following molar ratios: 20:1, 10:1, 5:1, 3:1, 2:1, 1:1 or 1:2.

In addition, it may be desirable to provide CO and H2 in particular ratios at different stages of the fermentation. For example, substrate streams with a relatively high H2 content (such as 1:2 CO:H2) may be provided to the fermentation stage during start up and/or phases of rapid microbial growth. However, when the growth phase slows, such that the culture is maintained at a substantially steady microbial density, the CO content may be increased (such as at least 1:1 or 2:1 or higher, wherein the H2 concentration may be greater or equal to zero).

Blending of streams may also have further advantages, particularly in instances where a waste stream comprising CO is intermittent in nature. For example, an intermittent waste stream comprising CO may be blended with a substantially continuous reformed substrate stream comprising CO and H2 and provided to the fermenter. In particular embodiments of the invention, the composition and flow rate of the substantially continuous blended stream may be varied in accordance with the intermittent stream in order to maintain provision of a substrate stream of substantially continuous composition and flow rate to the fermenter.

Blending of two or more streams to achieve a desirable composition may involve varying flow rates of all streams, or one or more of the streams may be maintained constant while other stream(s) are varied in order to ‘trim’ or optimise the blended stream to the desired composition. For streams that are processed continuously, little or no further treatment (such as buffering) may be necessary and the stream can be provided to the fermenter directly. However, it may be necessary to provide buffer storage for streams where one or more is available intermittently, and/or where streams are available continuously, but are used and/or produced at variable rates.

Those skilled in the art will appreciate it will be necessary to monitor the composition and flow rates of the streams prior to blending. Control of the composition of the blended stream can be achieved by varying the proportions of the constituent streams to achieve a target or desirable composition. For example, a base load gas may be predominantly CO and H2 of a particular ratio, and a secondary gas comprising a high concentration of CO may be blended to achieve a specified H2:CO ratio. The composition and flow rate of the blended stream can be monitored by any means known in the art. The flow rate of the blended stream can be controlled independently of the blending operation; however the rates at which the individual constituent streams can be drawn must be controlled within limits. For example, a stream produced intermittently, drawn continuously from buffer storage, must be drawn at a rate such that buffer storage capacity is neither depleted nor filled to capacity.

At the point of blending, the individual constituent gases will enter a mixing chamber, which will typically be a small vessel, or a section of pipe. In such cases, the vessel or pipe may be provided with static mixing devices, such as baffles, arranged to promote turbulence and rapid homogenisation of the individual components.

Buffer storage of the blended stream can also be provided if necessary, in order to maintain provision of a substantially continuous substrate stream to the bioreactor.

A processor adapted to monitor the composition and flow rates of the constituent streams and control the blending of the streams in appropriate proportions, to achieve the required or desirable blend may optionally be incorporated into the system. For example, particular components may be provided in an as required or an as available manner in order to optimise the efficiency of alcohol productivity and/or overall carbon capture.

It may not be possible or cost effective to provide CO and H2 at a particular ratio all the time. As such, a system adapted to blend two or more streams as described above may be adapted to optimise the ratio with the available resources. For example, in instances where an inadequate supply of H2 is available, the system may include means to divert excess CO away from the system in order to provide an optimised stream and achieve improved efficiency in alcohol production and/or overall carbon capture. In certain embodiments of the invention, the system is adapted to continuously monitor the flow rates and compositions of at least two streams and combine them to produce a single blended substrate stream of optimal composition, and means for passing the optimised substrate stream to the fermenter. In particular embodiments employing carboxydotrophic microbes to produce alcohol, the optimum composition of substrate stream comprising at least 1% H2 and up to about 1:2 CO:H2.

By way of non limiting example, particular embodiments of the invention involve the utilisation of converter gas from the decarburisation of steel as a source of CO. Typically, such streams contain little or no H2, therefore it may be desirable to combine the stream comprising CO with a reformed substrate stream comprising CO and H2 in order to achieve a more desirable CO:H2 ratio.

Additionally, or alternatively, a gasifier may be provided to produce CO and H2 from a variety of sources. The stream produced by the gasifier may be blended with a reformed substrate stream comprising CO and H2 to achieve a desirable composition. Those skilled in the art will appreciate that gasifier conditions can be controlled to achieve a particular CO:H2 ratio. Furthermore, the gasifier may be ramped up and down to increase and decrease the flow rate of the reformed substrate stream comprising CO and H2 produced by the gasifier. Accordingly, a stream from a gasifier may be blended with a substrate stream comprising CO and H2 to optimise the CO:H2 ratio in order to increase alcohol productivity and/or overall carbon capture. Furthermore, the gasifier may be ramped up and down to provide a stream of varying flow and/or composition that may be blended with an intermittent stream comprising CO and H2 to achieve a substantially continuous stream of desirable composition.

### Fermentation Reaction

Particular embodiments of the invention include the fermentation of a syngas substrate stream to produce products including alcohol(s) and optionally acid(s). Processes for the production of ethanol and other alcohols from gaseous substrates are known. Exemplary processes include those described for example in WO2007/117157, WO2008/115080, U.S. Pat. No. 6,340,581, U.S. Pat. No. 6,136,577, U.S. Pat. No. 5,593,886, U.S. Pat. No. 5,807,722 and U.S. Pat. No. 5,821,111, each of which is incorporated herein by reference.

A number of anaerobic bacteria are known to be capable of carrying out the fermentation of CO to alcohols, including n-butanol and ethanol, and acetic acid, and are suitable for use in the process of the present invention. Examples of such bacteria that are suitable for use in the invention include those of the genus Clostridium, such as strains of Clostridium ljungdahlii, including those described in WO 00/68407, EP 117309, U.S. Pat. Nos. 5,173,429, 5,593,886, and 6,368,819, WO 98/00558 and WO 02/08438, Clostridium carboxydivorans (Liou et al., International Journal of Systematic and Evolutionary Microbiology 33: pp 2085-2091) and Clostridium autoethanogenum (Abrini et al, Archives of Microbiology 161: pp 345-351). Other suitable bacteria include those of the genus Moorella, including Moorella sp HUC22-1, (Sakai et al, Biotechnology Letters 29: pp 1607-1612), and those of the genus Carboxydothermus (Svetlichny, V. A., Sokolova, T. G. et al (1991), Systematic and Applied Microbiology 14: 254-260). Further examples include Morella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa et. al. Critical Reviews in Biotechnology, 2006 Vol. 26. Pp 41-65). In addition, it should be understood that other acetogenic anaerobic bacteria may be applicable to the present invention as would be understood by a person of skill in the art. It will also be appreciated that the invention may be applied to a mixed culture of two or more bacteria.

One exemplary micro-organism suitable for use in the present invention is Clostridium autoethanogenum. In one embodiment, the Clostridium autoethanogenum is a Clostridium autoethanogenum having the identifying characteristics of the strain deposited at the German Resource Centre for Biological Material (DSMZ) under the identifying deposit number 19630. In another embodiment, the Clostridium autoethanogenum is a Clostridium autoethanogenum having the identifying characteristics of DSMZ deposit number DSMZ 10061. In another embodiment the Clostridium autoethanogenum is a Clostridium autoethanogenum having the identifying characteristics of DSMZ deposit number DSMZ 23693. Examples of fermentation of a substrate comprising CO to produce products including alcohols by Clostridium autoethanogenum are provided in WO2007/117157, WO2008/115080, WO2009/022925, WO2009/058028, WO2009/064200, WO2009/064201, WO2009/113878 and WO2009/151342 all of which are incorporated herein by reference.

Culturing of the bacteria used in the methods of the invention may be conducted using any number of processes known in the art for culturing and fermenting substrates using anaerobic bacteria. Exemplary techniques are provided in the “Examples” section below. By way of further example, those processes generally described in the following articles using gaseous substrates for fermentation may be utilised: (i) K. T. Klasson, et al. (1991). Bioreactors for synthesis gas fermentations resources. Conservation and Recycling, 5; 145-165; (ii) K. T. Klasson, et al. (1991). Bioreactor design for synthesis gas fermentations. Fuel. 70. 605-614; (iii) K. T. Klasson, et al. (1992). Bioconversion of synthesis gas into liquid or gaseous fuels. Enzyme and Microbial Technology. 14; 602-608; (iv) J. L. Vega, et al. (1989). Study of Gaseous Substrate Fermentation: Carbon Monoxide Conversion to Acetate. 2. Continuous Culture. Biotech. Bioeng. 34. 6. 785-793; (vi) J. L. Vega, et al. (1989). Study of gaseous substrate fermentations: Carbon monoxide conversion to acetate. 1. Batch culture. Biotechnology and Bioengineering. 34. 6. 774-784; (vii) J. L. Vega, et al. (1990). Design of Bioreactors for Coal Synthesis Gas Fermentations. Resources, Conservation and Recycling. 3. 149-160; all of which are incorporated herein by reference.

The fermentation may be carried out in any suitable bioreactor configured for gas/liquid contact wherein the substrate can be contacted with one or more microorganisms, such as a continuous stirred tank reactor (CSTR), an immobilised cell reactor, a gas-lift reactor, a bubble column reactor (BCR), a membrane reactor, such as a Hollow Fibre Membrane Bioreactor (HFMBR) or a trickle bed reactor (TBR), monolith bioreactor or loop reactors. Also, in some embodiments of the invention, the bioreactor may comprise a first, growth reactor in which the micro-organisms are cultured, and a second, fermentation reactor, to which fermentation broth from the growth reactor is fed and in which most of the fermentation product (e.g. ethanol and acetate) is produced.

According to various embodiments of the invention, the carbon source for the fermentation reaction is syngas derived from gasification. The syngas substrate will typically contain a major proportion of CO, such as at least about 15% to about 75% CO by volume, from 20% to 70% CO by volume, from 20% to 65% CO by volume, from 20% to 60% CO by volume, and from 20% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when H₂ and CO₂ are also present. In particular embodiments, the presence of hydrogen results in an improved overall efficiency of alcohol production. The gaseous substrate may also contain some CO₂ for example, such as about 1% to about 80% CO₂ by volume, or 1% to about 30% CO₂ by volume.

In accordance with particular embodiments of the invention, the CO content and/or the H2 content of the reformed substrate stream can be enriched prior to passing the stream to the bioreactor. For example, hydrogen can be enriched using technologies well known in the art, such as pressure swing adsorption, cryogenic separation and membrane separation. Similarly, CO can be enriched using technologies well known in the art, such as copper-ammonium scrubbing, cryogenic separation, COSORB™ technology (absorption into cuprous aluminium dichloride in toluene), vacuum swing adsorption and membrane separation. Other methods used in gas separation and enrichment are detailed in PCT/NZ2008/000275, which is fully incorporated herein by reference.

Typically, the carbon monoxide will be added to the fermentation reaction in a gaseous state. However, the methods of the invention are not limited to addition of the substrate in this state. For example, the carbon monoxide can be provided in a liquid. For example, a liquid may be saturated with a carbon monoxide containing gas and that liquid added to the bioreactor. This may be achieved using standard methodology. By way of example a microbubble dispersion generator (Hensirisak et. al. Scale-up of microbubble dispersion generator for aerobic fermentation; Applied Biochemistry and Biotechnology Volume 101, Number 3/October, 2002) could be used for this purpose.

It will be appreciated that for growth of the bacteria and CO-to-alcohol fermentation to occur, in addition to the CO-containing substrate gas, a suitable liquid nutrient medium will need to be fed to the bioreactor. A nutrient medium will contain vitamins and minerals sufficient to permit growth of the micro-organism used. Anaerobic media suitable for the fermentation of ethanol using CO as the sole carbon source are known in the art. For example, suitable media are described in U.S. Pat. Nos. 5,173,429 and 5,593,886 and WO 02/08438, WO2007/117157, WO2008/115080, WO2009/022925, WO2009/058028, WO2009/064200, WO2009/064201, WO2009/113878 and WO2009/151342 referred to above. The present invention provides a novel media which has increased efficacy in supporting growth of the micro-organisms and/or alcohol production in the fermentation process. This media will be described in more detail hereinafter.

The fermentation should desirably be carried out under appropriate conditions for the desired fermentation to occur (e.g. CO-to-ethanol). Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition. Suitable conditions are described in WO02/08438, WO2007/117157, WO2008/115080, WO2009/022925, WO2009/058028, WO2009/064200, WO2009/064201, WO2009/113878 and WO2009/151342 all of which are incorporated herein by reference.

The optimum reaction conditions will depend partly on the particular micro-organism used. However, in general, it is preferred that the fermentation be performed at pressure higher than ambient pressure. Operating at increased pressures allows a significant increase in the rate of CO transfer from the gas phase to the liquid phase where it can be taken up by the micro-organism as a carbon source for the production of ethanol. This in turn means that the retention time (defined as the liquid volume in the bioreactor divided by the input gas flow rate) can be reduced when bioreactors are maintained at elevated pressure rather than atmospheric pressure.

The benefits of conducting a gas-to-ethanol fermentation at elevated pressures have also been described elsewhere. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO and H2 containing gaseous substrate is such as to ensure that the concentration of CO in the liquid phase does not become limiting. This is because a consequence of CO-limited conditions may be that the ethanol product is consumed by the culture.

### Product Recovery

The products of the fermentation reaction can be recovered using known methods. Exemplary methods include those described in WO2007/117157, WO2008/115080, WO2009/022925, U.S. Pat. No. 6,340,581, U.S. Pat. No. 6,136,577, U.S. Pat. No. 5,593,886, U.S. Pat. No. 5,807,722 and U.S. Pat. No. 5,821,111. However, briefly and by way of example only ethanol may be recovered from the fermentation broth by methods such as fractional distillation or evaporation, and extractive fermentation.

Distillation of ethanol from a fermentation broth yields an azeotropic mixture of ethanol and water (i.e., 95% ethanol and 5% water). Anhydrous ethanol can subsequently be obtained through the use of molecular sieve ethanol dehydration technology, which is also well known in the art.

Extractive fermentation procedures involve the use of a water-miscible solvent that presents a low toxicity risk to the fermentation organism, to recover the ethanol from the dilute fermentation broth. For example, oleyl alcohol is a solvent that may be used in this type of extraction process. Oleyl alcohol is continuously introduced into a fermenter, whereupon this solvent rises forming a layer at the top of the fermenter which is continuously extracted and fed through a centrifuge. Water and cells are then readily separated from the oleyl alcohol and returned to the fermenter while the ethanol-laden solvent is fed into a flash vaporization unit. Most of the ethanol is vaporized and condensed while the oleyl alcohol is non volatile and is recovered for re-use in the fermentation.

Acetate, which is produced as by-product in the fermentation reaction, may also be recovered from the fermentation broth using methods known in the art.

For example, an adsorption system involving an activated charcoal filter may be used. In this case, it is preferred that microbial cells are first removed from the fermentation broth using a suitable separation unit. Numerous filtration-based methods of generating a cell free fermentation broth for product recovery are known in the art. The cell free ethanol—and acetate—containing permeate is then passed through a column containing activated charcoal to adsorb the acetate. Acetate in the acid form (acetic acid) rather than the salt (acetate) form is more readily adsorbed by activated charcoal. It is therefore preferred that the pH of the fermentation broth is reduced to less than about 3 before it is passed through the activated charcoal column, to convert the majority of the acetate to the acetic acid form.

Acetic acid adsorbed to the activated charcoal may be recovered by elution using methods known in the art. For example, ethanol may be used to elute the bound acetate. In certain embodiments, ethanol produced by the fermentation process itself may be used to elute the acetate. Because the boiling point of ethanol is 78.8° C. and that of acetic acid is 107° C., ethanol and acetate can readily be separated from each other using a volatility-based method such as distillation.

Other methods for recovering acetate from a fermentation broth are also known in the art and may be used in the processes of the present invention. For example, U.S. Pat. Nos. 6,368,819 and 6,753,170 describe a solvent and cosolvent system that can be used for extraction of acetic acid from fermentation broths. As with the example of the oleyl alcohol-based system described for the extractive fermentation of ethanol, the systems described in U.S. Pat. Nos. 6,368,819 and 6,753,170 describe a water immiscible solvent/co-solvent that can be mixed with the fermentation broth in either the presence or absence of the fermented micro-organisms in order to extract the acetic acid product. The solvent/co-solvent containing the acetic acid product is then separated from the broth by distillation. A second distillation step may then be used to purify the acetic acid from the solvent/co-solvent system.

The products of the fermentation reaction (for example ethanol and acetate) may be recovered from the fermentation broth by continuously removing a portion of the broth from the fermentation bioreactor, separating microbial cells from the broth (conveniently by filtration), and recovering one or more product from the broth simultaneously or sequentially. In the case of ethanol it may be conveniently recovered by distillation, and acetate may be recovered by adsorption on activated charcoal, using the methods described above. The separated microbial cells are preferably returned to the fermentation bioreactor. The cell free permeate remaining after the ethanol and acetate have been removed is also preferably returned to the fermentation bioreactor. Additional nutrients (such as B vitamins) may be added to the cell free permeate to replenish the nutrient medium before it is returned to the bioreactor. Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

### General

Embodiments of the invention are described by way of example. However, it should be appreciated that particular steps or stages necessary in one embodiment may not be necessary in another. Conversely, steps or stages included in the description of a particular embodiment can be optionally advantageously utilised in embodiments where they are not specifically mentioned.

While the invention is broadly described with reference to any type of stream that may be moved through or around the system(s) by any known transfer means, in certain embodiments, the biogas and reformed and/or blended substrate streams are gaseous. Those skilled in the art will appreciate that particular stages may be coupled by suitable conduit means or the like, configurable to receive or pass streams throughout a system. A pump or compressor may be provided to facilitate delivery of the streams to particular stages. Furthermore, a compressor can be used to increase the pressure of gas provided to one or more stages, for example the bioreactor. As discussed hereinabove, the pressure of gases within a bioreactor can affect the efficiency of the fermentation reaction performed therein. Thus, the pressure can be adjusted to improve the efficiency of the fermentation. Suitable pressures for common reactions are known in the art.

In addition, the systems or processes of the invention may optionally include means for regulating and/or controlling other parameters to improve overall efficiency of the process. For example particular embodiments may include determining means to monitor the composition of substrate and/or exhaust stream(s). In addition, particular embodiments may include a means for controlling the delivery of substrate stream(s) to particular stages or elements within a particular system if the determining means determines the stream has a composition suitable for a particular stage. For example, in instances where a gaseous substrate stream contains low levels of CO or high levels of O₂ that may be detrimental to a fermentation reaction, the substrate stream may be diverted away from the bioreactor. In particular embodiments of the invention, the system includes means for monitoring and controlling the destination of a substrate stream and/or the flow rate, such that a stream with a desired or suitable composition can be delivered to a particular stage.

In addition, it may be necessary to heat or cool particular system components or substrate stream(s) prior to or during one or more stages in the process. In such instances, known heating or cooling means may be used.

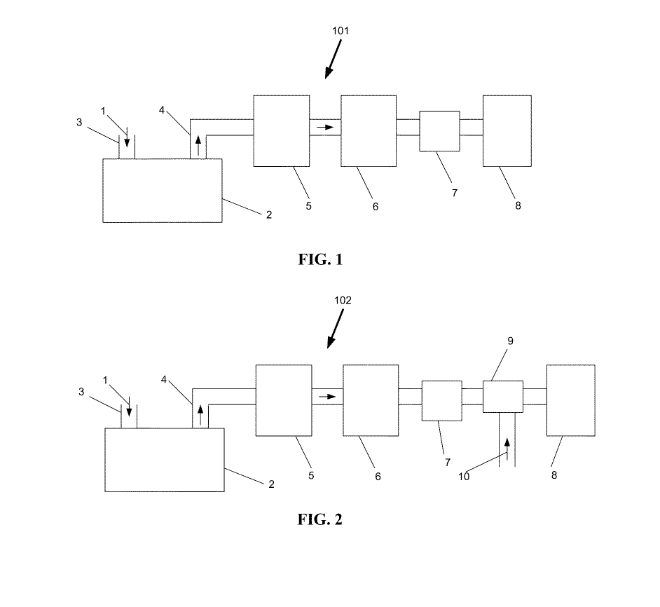

Various embodiments of the systems of the invention are described in the accompanying Figures. The alternative embodiments described in FIGS. 1 and 2 comprise features in common with one another and the same reference numbers have been used to denote the same or similar features in the various figures. Only the new features (relative to FIG. 1) of FIG. 2 are described, and so these Figures should be considered in conjunction with the description of FIG. 1.

FIG. 1 is a schematic representation of a system 101 according to one embodiment of the invention. Biodegradable material 1 is fed into anaerobic digester 2 via inlet port 3. The digester 2 is maintained under anaerobic conditions, wherein the biodegradable material is digested to produce a biogas stream comprising methane. Conditions within the digester 2 can be optimised by addition or removal of particular components, and/or altering of particular parameters. For example, heating or cooling the digester 2, addition of water, removal of waste liquid. The biogas produced exits the digester 2 by exit port 4, where it is passed to optional separator 5. The optional separator 5 is configured to remove one or more components of the biogas stream such as H2S, CO2, O2 and/or N2. The optionally conditioned gas is passed to the methane reformer 6, wherein CH4 is converted to a reformed substrate stream comprising CO and H2.

Pre-treat 7 may be used to control various aspects of the stream, including temperature and levels of contaminants or other undesired components or constituents. It may also be used to add components to the stream. This will depend on the particular composition of the syngas stream and/or the particular fermentation reaction and/or the micro-organisms selected therefor.

Pre-treat 7 may be positioned elsewhere within system 101 or may be omitted, or multiple pre-treats 7 may be provided at various points in system 101. This will depend on the particular source of the biogas and/or substrate stream and/or the particular fermentation reaction and/or the micro-organisms selected therefor.

Following optional pre-treatment the reformed substrate stream may be passed to bioreactor 8 by any known transfer means. Bioreactor 8 is configured to perform the desired fermentation reaction to produce products. According to certain embodiments, bioreactor 8 is configured to process a CO and H2 containing substrate so as to produce one or more acids and/or one or more alcohols by microbial fermentation. In a particular embodiment, bioreactor 8 is used to produce ethanol and/or butanol. Bioreactor 8 may comprise more than one tank, each tank being configured to perform the same reaction and/or different stages within a particular fermentation process and/or different reactions, including different reactions for different fermentation processes which may include one or more common stages.

Bioreactor 8 may be provided with cooling means for controlling the temperature therein within acceptable limits for the micro-organisms used in the particular fermentation reaction to be performed.

A pump or compressor (not shown) may be provided upstream of bioreactor 8 so that the pressure of gas within bioreactor 8 is increased. As discussed hereinabove, the pressure of gases within a bioreactor can affect the efficiency of the fermentation reaction performed therein. Thus, the pressure can be adjusted to improve the efficiency of the fermentation. Suitable pressures for common reactions are known in the art.

The products produced in the bioreactor 8 may be recovered by any recovery process known in the art.

FIG. 2 is a schematic representation of a system 102 according to another embodiment of the invention. System 102 includes blending means to blend one or more additional streams 10, such as waste streams from an industrial process. In particular embodiments, the blending means 10 includes a mixing chamber which will typically comprise a small vessel or a section of pipe. In such cases, the vessel or pipe may be provided with mixing means, such as baffles, adapted to promote turbulence and rapid homogenisation of the individual components.

In certain embodiments of the invention, the blending means 10 includes means for controlling the blending of two or more streams to achieve a desirable optimised substrate stream. For example, the blending means 10 may include means to control the flow rates of each of the streams entering the blending means 10 such that a desirable composition of the blended stream is achieved. (e.g. desirable CO:H2 ratio) The blender also preferably includes monitoring means (continuous or otherwise) downstream of the mixing chamber. In particular embodiments, the blender includes a processor adapted to control the flow rates and/or compositions of the various streams as a result of feedback from the monitoring means.

Means for determining the composition of the stream may be optionally included at any stage of the system. Such means can be associated with diverting means such that streams with particular compositions can be diverted to or away from particular stages if necessary or as desired. Means for diverting and/or transferring the streams around the various stages of the system will be known to those skilled in the art.

### EXAMPLES

### Media Preparation

Solution A

| | | | | | |
|---|---|---|---|---|---|
| NH₄Ac | 3.083 | g | KCl | 0.15 | g |
| MgCl₂•6H₂O | 0.61 | g | NaCl | 0.12 | g |

| | | | | |
|---|---|---|---|---|
| CaCl₂•2H₂O | 0.294 | g | Distilled Water | Up to 1 L |

Solution B

| Component/ | Component/ | | Component/ | Component/ | |
|---|---|---|---|---|---|
| 0.1M solution | 0.1M solution | | 0.1M solution | 0.1M solution | |
| (aq) | (aq) | | (aq) | (aq) | |
| Component/ | Quantity/ | | Component/ | Quantity/ | |
| 0.1M | ml into | | 0.1M | ml into | |
| FeCl₃ | 1 | ml | Na₂WO₄ | 0.1 | ml |
| CoCl₂ | 0.5 | ml | ZnCl₂ | 0.1 | ml |
| NiCl₂ | 0.5 | ml | Na₂MoO₄ | 0.1 | ml |
| H₃BO₃ | 0.1 | ml | | | |

Solution C

| Biotin | 20.0 | mg | Calcium D-(*)- | 50.0 | mg |
|---|---|---|---|---|---|
| | | | pantothenate | | |
| Folic acid | 20.0 | mg | Vitamin B12 | 50.0 | mg |
| Pyridoxine•HCl | 10.0 | mg | p-Aminobenzoic | 50.0 | mg |
| Thiamine•HCl | 50.0 | mg | Thioctic acid | 50.0 | mg |
| Riboflavin | 50.0 | mg | Distilled water | To 1 Litre | |
| Nicotinic acid | 50.0 | mg | | | |

Solution D

| | | | | | |
|---|---|---|---|---|---|
| NH₄Ac | 3.083 | g | KCl | 0.15 | g |
| MgCl₂•6H₂O | 0.407 | g | NaCl | 0.12 | g |

| | | | | |
|---|---|---|---|---|
| CaCl₂•2H₂O | 0.294 | g | Distilled Water | Up to 1 L |

Solution E

| | | | | | |
|---|---|---|---|---|---|
| MgCl₂•6H₂O | 0.407 | g | KCl | 0.15 | g |

| | | | | |
|---|---|---|---|---|
| CaCl₂•2H₂O | 0.294 | g | Distilled Water | Up to 1 L |

Solution F

| | | | | | |
|---|---|---|---|---|---|
| Solution D | 50 | ml | Solution E | 50 | ml |

### Bacteria:

In a preferred embodiment the Clostridium autoethanogenum is a Clostridium autoethanogenum having the identifying characteristics of the strain deposited at the German Resource Centre for Biological Material (DSMZ) under the identifying deposit number 10061. In another embodiment the Clostridium autoethanogenum is a Clostridium autoethanogenum having the identifying characteristics of DSMZ deposit number DSMZ 23693.

### Sampling and Analytical Procedures

Media samples were taken from the CSTR reactor at intervals over periods up to 10 days. Each time the media was sampled care was taken to ensure that no gas was allowed to enter into or escape from the reactor.

### HPLC:

HPLC System Agilent 1100 Series. Mobile Phase: 0.0025N Sulfuric Acid. Flow and pressure: 0.800 mL/min. Column: Alltech IOA; Catalog #9648, 150×6.5 mm, particle size 5 μm. Temperature of column: 60° C. Detector: Refractive Index. Temperature of detector: 45° C.

### Method for Sample Preparation:

400 μL of sample and 50 μL of 0.15M ZnSO₄ are mixed and loaded into an Eppendorf tube. The tubes are centrifuged for 3 min. at 12,000 rpm, 4° C. 200 μL of the supernatant are transferred into an HPLC vial, and 5 μL are injected into the HPLC instrument.

### Gas Chromatography:

Gas Chromatograph HP 5890 series II utilizing a Flame Ionization Detector. Capillary GC Column: EC1000—Alltech EC1000 30m×0.25 mm×0.25 nm. The Gas Chromatograph was operated in Split mode with a total flow of hydrogen of 50 mL/min with 5 mL purge flow (1:10 split), a column head pressure of 10 PSI resulting in a linear velocity of 45 cm/sec. The temperature program was initiated at 60° C., held for 1 minute then ramped to 215° C. at 30° C. per minute, then held for 2 minutes. Injector temperature was 210° C. and the detector temperature was 225° C.

### Method for Sample Preparation:

500 μL sample is centrifuged for 10 min at 12,000 rpm, 4° C. 100 μL of the supernatant is transferred into an GC vial containing 200 μL water and 100 μL of internal standard spiking solution (10 g/L propan-1-ol, 5 g/L iso-butyric acid, 135 mM hydrochloric acid). 1 μL of the solution is injected into the GC instrument.

### Cell Density:

Cell density was determined by counting bacterial cells in a defined aliquot of fermentation broth. Alternatively, the absorbance of the samples was measured at 600 nm (spectrophotometer) and the dry mass determined via calculation according to published procedures.

### Example 1

### Serum Bottles

1.9 litres of media solution A was aseptically and anaerobically transferred into a 2 L CSTR vessel, and continuously sparged with N₂. Once transferred to the fermentation vessel, the reduction state and pH of the transferred media could be measured directly via probes. The media was heated to 37° C. and stirred at 400 rpm and 1.5 ml of resazurin (2 g/L) was added. 1.0 ml of H3P04 85% was added to obtain a 10 mM solution. 2 g ammonium acetate was added and the pH was adjusted to 5.3 using NH4OH.

NTA (0.15M) was added to five a final concentration of 0.03 mM. Metal ions were added according to solution B and 15 ml of solution C was added. 3 mmol cysteine was added and the pH was adjusted to pH 5.5 using NH4OH.

Incubation was performed in three 250 ml sealed serum bottles (SB1, SB2 and SB3) containing 50 ml of the media. Each bottle was inoculated with lml of a growing culture of Clostridium autoethanogenum (DSMZ number 23693). The headspace gas was then pressurised to 30 psig with a gas mixture having the following composition; CO₂ 5%, CO 17%, H₂ 70% and N₂ 2.5%. A shaking incubator was used and the reaction temperature was maintained at 37° C.

### Results

TABLE 1
Metabolite measurements (g/L)

| Sample | | incubation | | | 2,3 | lactic |
|---|---|---|---|---|---|---|
| no. | Date | time | Acetate | Ethanol | BDO | acid |
| SB1 | 22/04/2011 17:35 | 0.0 | 1.01 | 0.18 | 0.03 | 0 |
| SB2 | 22/04/2011 17:36 | 0.0 | 1.02 | 0.17 | 0.02 | 0 |
| SB3 | 22/04/2011 18:35 | 0.0 | 1.02 | 0.16 | 0.03 | 0 |
| SB1 | 25/04/2011 15:33 | 2.9 | 1.47 | 0.32 | 0.03 | 0 |
| SB2 | 25/04/2011 15:33 | 2.9 | 1.73 | 0.61 | 0.03 | 0 |
| SB3 | 25/04/2011 15:33 | 2.9 | 1.7 | 0.74 | 0.03 | 0 |

TABLE 2
Gas concentrations (% by volume)

| | | |
|---|---|---|
| Sample | Incubation | Gas Composition |

| Number | Time | CO₂ | CO | H₂ | N₂ |
|---|---|---|---|---|---|
| SB2 | 2.9 | 14.0% | 0.04% | 82.6% | 2.5% |
| SB3 | 2.9 | 15.11% | 0.0% | 81.3% | 2.5% |

Table 1 shows the results for the three serum bottles. The table shows the metabolites measurements immediately after inoculation and results at day 2.9. Table 2 shows the gas composition in the headspace at day 2.9. The results clearly show utilisation of CO. SB2 shows a decrease in CO % from 17% to 0.04% and an increase in CO₂ from 5% to 14.0%. SB3 demonstrates utilisation of all of the CO introduced to the serum bottle, and an increase in CO₂ from 5% to 15.11%. The gas composition in SB1 was not measured. Correspondingly all three serum bottles show an increase in the metabolite levels between day 0.0 and day 2.9. The above results demonstrate the fermentation of CO by C autoethanogenum to produce ethanol and acetate.

### Example 2

### Serum Bottles Using Gaseous Substrate Derived from Landfill Biogas

### Gaseous Substrate

The biogas source for the gaseous substrate for this experiment was derived from landfill biogas. The land fill biogas had a composition as follows;

### CH₄ 71.86%, CO2 7.38%, N2 17.83% O2 2.93%.

The biogas was converted to gaseous substrate comprising CO by a steam reforming process. The steam reforming was carried out in an Inconel® 800 reactor at a temperature of around 818° C. and a temperature of around 128 psig. The reactor was loaded with a nickel-alumina catalyst and a steam to carbon ration (S/C) of 3.6 was used for the biogas reforming. Prior to the reforming process, the biogas was blended with CO₂ to obtain a CH₄/CO₂ ratio of about 1.5.

Steam reforming of the biogas resulted in a gaseous substrate having the following composition;

CH₄ 0.3%; CO₂ 19.1%; CO 14; H₂ 62.5%, N₂ 5.0%

### Innoculum Preparation

4 litres of distilled H₂O was aseptically and anaerobically transferred into a 5 L CSTR vessel. 100 ml of solution E was added and the vessel was continuously sparged with N₂. Once transferred to the fermentation vessel, the reduction state and pH of the transferred media could be measured directly via probes. The media was heated to 37° C. and stirred at 400 rpm and 2.5 ml of resazurin (2 g/L) was added. 1.875 ml of H3P04 85% was added.

Metal ions were added according to solution B and 50 ml of solution C was added. 2.5 g of Cysteine (3 mM) was added and the pH was adjusted to 5.3 using NH4OH.

400 ml of an actively growing Clostridium autoethanogenum culture was inoculated into the CSTR. During these experiments, the pH was adjusted and/or maintained by a controller through the automated addition of buffers (0.5 M NaOH or 2N H₂SO₄).

### Serum Bottle Preparation and Innoculation

Two 250 ml serum bottles were inoculated with 50 mls of a live culture of Clostridium autoethanogenum as prepared above.

The headspace gas was then pressurised to 24 psig with the reformed biogas mixture.

A shaking incubator was used and the reaction temperature was maintained at 37° C.

### Results

TABLE 3
Metabolite measurements (g/L)

| | | incubation | | | | |
|---|---|---|---|---|---|---|
| Sample | | time | Ace- | | 2,3 | Lactic |
| no. | Date | (days) | tate | Ethanol | BDO | acid |
| SB1 | 3/05/2011 11:28 | 0.0 | 0.69 | 1.91 | 0.22 | 0.05 |
| SB2 | 3/05/2011 11:28 | 0.0 | 0.68 | 2.15 | 0.22 | 0.05 |
| SB1 | 3/05/2011 16:19 | 0.2 | 1.06 | 2.22 | 0.27 | 0.04 |
| SB2 | 3/05/2011 16:19 | 0.2 | 1.00 | 2.53 | 0.28 | 0.05 |
| SB1 | 4/05/2011 8:32 | 0.7 | 1.07 | 2.25 | 0.27 | 0.05 |
| SB2 | 4/05/2011 8:32 | 0.7 | 1.01 | 2.52 | 0.29 | 0.05 |

Table 3 shows the results for the two serum bottles. The table shows the metabolites measurements immediately after inoculation and results at day 2.9.

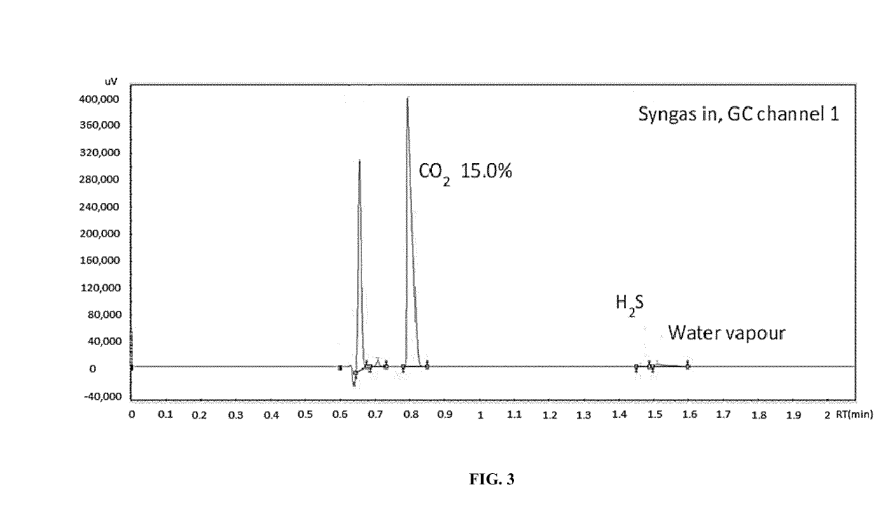

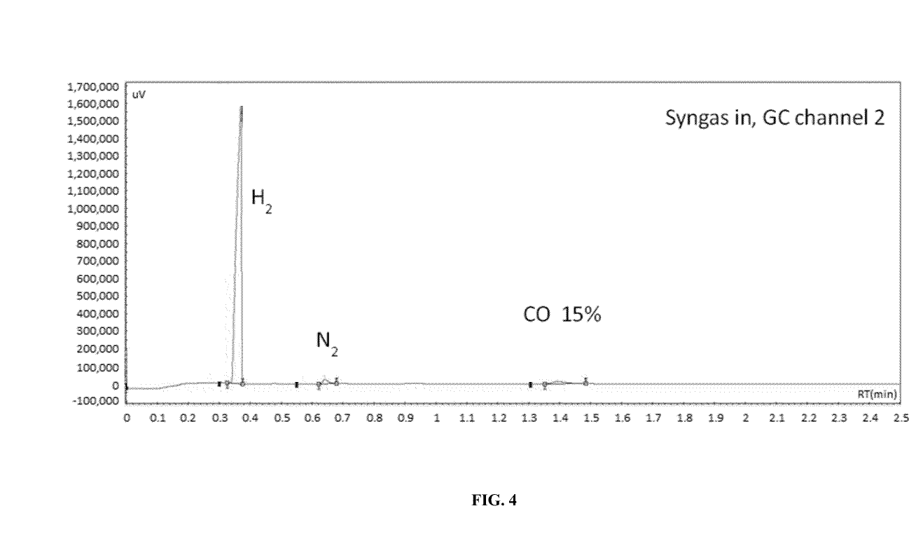

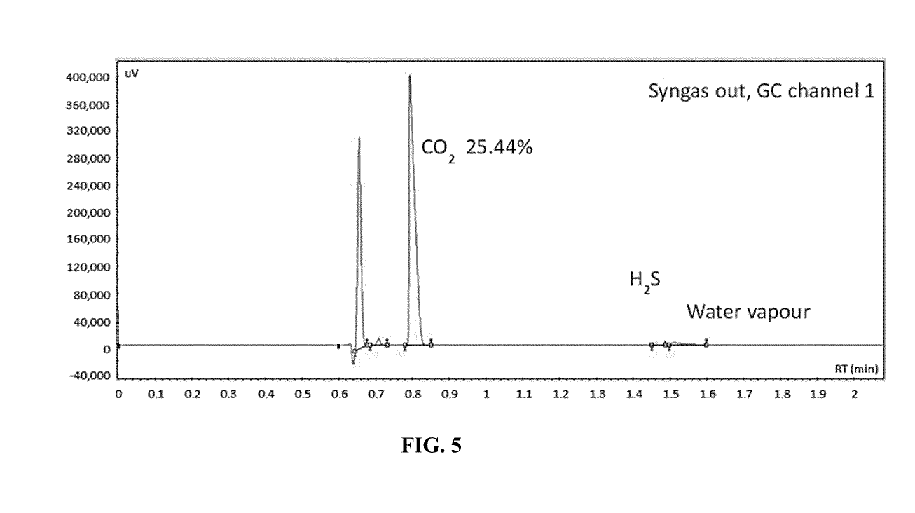

FIGS. 3 and 4 demonstrate the gas composition in the headspace of the serum bottles at day 0.0. FIGS. 3 and 4 demonstrate a CO concentration of 15% and a CO₂ concentration of 15%.

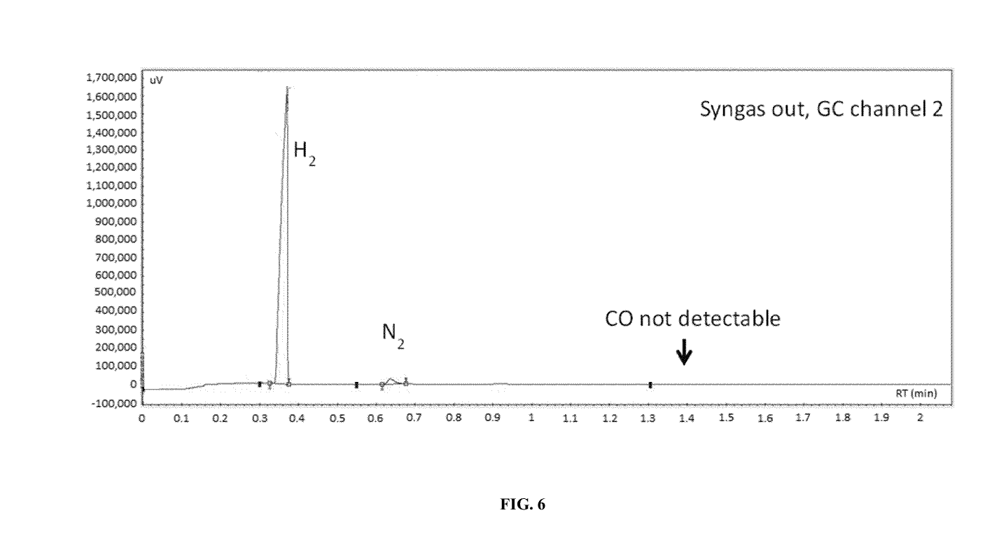

FIGS. 5 and 6 demonstrate the gas composition in the headspace of the serum bottles at day 0.7. As seen in FIG. 5, the CO₂ concentration increases to 25.44%. The CO concentration is FIG. 6 is not detectable, clearly demonstrating utilisation of CO by fermentation with Clostridium autoethanogenum.

The invention has been described herein with reference to certain preferred embodiments, in order to enable the reader to practice the invention without undue experimentation. Those skilled in the art will appreciate that the invention can be practiced in a large number of variations and modifications other than those specifically described. It is to be understood that the invention includes all such variations and modifications. Furthermore, titles, heading, or the like are provided to aid the reader's comprehension of this document, and should not be read as limiting the scope of the present invention. The entire disclosures of all applications, patents and publications cited herein are herein incorporated by reference.

More particularly, as will be appreciated by one of skill in the art, implementations of embodiments of the invention may include one or more additional elements. Only those elements necessary to understand the invention in its various aspects may have been shown in a particular example or in the description. However, the scope of the invention is not limited to the embodiments described and includes systems and/or methods including one or more additional steps and/or one or more substituted steps, and/or systems and/or methods omitting one or more steps.

The reference to any prior art in this specification is not, and should not be taken as, an acknowledgement or any form of suggestion that that prior art forms part of the common general knowledge in the field of endeavour in any country.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

1. A method for producing at least one product by microbial fermentation, the method comprising:
a) passing a biogas stream comprising methane to a steam reforming zone, operated at reforming conditions whereby at least a portion of the methane in the biogas stream is converted to a substrate comprising CO and H₂; and
b) passing the substrate to a bioreactor comprising a culture of at least one carboxydotrophic microorganisms, and anaerobically fermenting the substrate to at least one product selected from the group consisting of alcohols, acids and mixtures thereof.

2. The method of claim 1 where the biogas stream further comprises methane at least one component selected from the group consisting of CO₂, N₂, H₂, H₂S and O₂.

3. The method of claim 2 where the methane concentration in the biogas stream is enriched by removing at least a portion of at least one component prior to passing the biogas to the steam reforming zone.

4. The method of claim 3 where the at least one component is removed in a pressure swing adsorption (PSA) zone.

5. The method of claim 1 further comprising admixing a gas stream comprising CO with the substrate to provide a blended substrate having a CO:H₂ molar ratio from about 20:1 to about 1:2.

6. The method of claim 1 where the alcohol is selected from the group consisting of ethanol, 2,3-butanediol and mixtures thereof.

7. The method of claim 1 where the acid is selected from the group consisting of acetate, lactic acid and mixtures thereof.

8. The method of claim 1 where the micro-organism is selected from the group consisting of Clostridium, Moorella, Pyrococcus, Eubacterium, Desulfobacterium, Carboxydothermus, Acetogenium, Acetobacterium, Acetoanaerobium, Butyribaceterium, Peptostreptococcus and mixtures thereof.

9. The method of claim 8 where the micro-organism is selected from the group consisting of Clostridium autoethanogenum, Clostridium ljungdahli, Clostridium ragsdalei, Clostridium carboxydivorans and mixtures thereof.

10. The method of claim 9 where the micro-organism is Clostridium autoethanogenum.

11. The method of claim 9 where the micro-organism is Clostridium autoethanogenum.

12. A method for producing at least one product by microbial fermentation, the method comprising:
a) passing a biogas stream comprising CH₄ and CO₂ to a pressure swing zone (PSA) operated at conditions to remove at least a portion of the CO₂ and provide a methane enriched biogas stream;
b) passing the methane enriched biogas stream to a steam reforming zone, operated at steam reforming conditions whereby at least a portion of the methane in the biogas stream is converted to a substrate comprising CO and H₂;
c) admixing a gas stream comprising CO with the substrate to provide a blended substrate having a CO:H₂ molar ratio from about 20:1 to about 1:2; and
d) passing the substrate to a bioreactor comprising a culture of at least one carboxydotrophic microorganisms, and anaerobically fermenting the substrate to at least one product selected from the group consisting of alcohols, acids and mixtures thereof.

13. The method of claim 12 where the alcohol is selected from the group consisting of ethanol, 2,3-butanediol and mixtures thereof.

14. The method of claim 12 where the acid is selected from the group consisting of acetate, lactic acid and mixtures thereof.

15. The method of claim 12 where the micro-organism is selected from the group consisting of Clostridium, Moorella, Pyrococcus, Eubacterium, Desulfobacterium, Carboxydothermus, Acetogenium, Acetobacterium, Acetoanaerobium, Butyribaceterium, Peptostreptococcus and mixtures thereof.

16. The method of claim 15 where the micro-organism is selected from the group consisting of Clostridium autoethanogenum, Clostridium ljungdahli, Clostridium ragsdalei, Clostridium carboxydivorans and mixtures thereof.

17. The method of claim 16 where the micro-organism is Clostridium autoethanogenum.

18. The method of claim 17 where the micro-organism is the Clostridium autoethanogenum strain deposited at the German Resource Centre for Biological Material (DSMZ) under the deposit number DSM 23693.

19. A method for producing at least one product by microbial fermentation, the method comprising:
a) passing a biogas stream comprising CH₄ and CO₂ to a pressure swing zone (PSA) operated at conditions to remove at least a portion of the CO₂ and provide a methane enriched biogas stream;
b) passing the methane enriched biogas stream to a steam reforming zone, operated at steam reforming conditions whereby at least a portion of the methane in the biogas stream is converted to a substrate comprising CO and H₂;
c) admixing a gas stream comprising CO with the substrate to provide a blended substrate having a CO:H₂ molar ratio from about 20:1 to about 1:2; and
d) passing the substrate to a bioreactor comprising a culture of Clostridium autoethanogenum and anaerobically fermenting the substrate to a product selected from the group consisting of ethanol, 2,3-butanediol, acetate, lactic acid and mixtures thereof.

20. The method of claim 19 where the micro-organism is the Clostridium autoethanogenum strain deposited at the German Resource Centre for Biological Material (DSMZ) under the deposit number DSM 23693.
